(19) United States
(12) Patent Application Publication
(10) Pub. No.: US 2015/0153317 A1
(43) Pub. Date: Jun. 4, 2015

(54) System for Inexpensive Characterization of Air Pollutants and Inexpensive Reduction of Indoor Dust

(71) Applicant: Acculation, Inc., Los Angeles, CA (US)

(72) Inventor: Werner Guether Krebs, Los Angeles, CA (US)

(73) Assignee: Acculation, Inc., Los Angeles, CA (US)

(21) Appl. No.: 14/547,140

(22) Filed: Nov. 19, 2014

Related U.S. Application Data
(63) Continuation-in-part of application No. 61/906,392, filed on Nov. 19, 2013.

(51) Int. Cl. G01N 33/00; B01D 46/42; G01W 1/00; B01D 46/44

(52) U.S. Cl. CPC G01N 33/0062; B01D 46/44; B01D 46/429; G01N 33/0036; G01W 1/00; B01D 2201/54

(57) ABSTRACT

The invention describes software (including mathematical models implemented in software) and related electronic circuits that can be used to combine data from local, inexpensive dust sensors (particle counters) with Internet-available rich data on pollutants, weather, optional household devices, sensors, and appliances to create a rich picture of the local environment, shape that environment through non-trivial control of said household appliances and ventilation systems to reduce buildup of household dust on surfaces or reduce sensitive individuals' exposure to specific pollutants, and monitor individuals' exposure to pollutants. The software might live in a smartphone (such as the inventors' iPhone prototype), related hardware devices (such as a pollution sensor communicating via bluetooth with the smartphone) or in heating/cooling control system such as a common household thermostat. In particular, advanced control of windows or inexpensive air filters within a common forced air climate system to mitigate air pollution inexpensively are envisioned.

### RELATED APPLICATIONS

The present application is a continuation-in-part application of U.S. provisional patent application, Ser. No. 61/906,392, filed Nov. 19, 2013, for METHOD FOR INEXPENSIVE CHARACTERIZATION OF AIR POLLUTANTS AND INEXPENSIVE REDUCTION OF INDOOR DUST, by Werner Guether Krebs, included by reference herein and for which benefit of the priority date is hereby claimed.

### FIELD OF THE INVENTION

The present invention relates to inexpensive air quality sensors and, more particularly, to systems for improving decision-making based on noisy data obtained from such inexpensive sensors by referencing 3rd party air quality data over a network, as well as improved circuitry and/or algorithms for decision making based on such readings within the larger context of home automation systems and typical home devices.

### BACKGROUND OF THE INVENTION

Particulate pollution remains a problem in many US cities and internationally (e.g., China). NASA estimates PM2.5 dust pollution kills more than 2 million annually, and it has been implicated in cancer, allergies, asthma, autism, not to mention household dust buildup and significant component in equipment failure. The most common measures of pollution are the PM2.5 and PM10 standards used by the EPA and other national and international authorities. These measure total micrograms per cubic meter of particulate pollutants below 2.5 microns and below 10 microns, respectively. It is generally believed that the body has fewer defenses against smaller particles, and thus the PM2.5 standard is generally believed to be the more important of the two measures. Smaller particles below 1 micron and especially below 0.1 microns are thought to be harder for the body to filter out, harder to dislodge once they enter internal organs such as the lungs, and more likely to pass from the lungs directly into the bloodstream. These smaller, dangerous particles are typically labeled ‘ultrafine’ and ‘nanoparticles’ and may include common, dangerous pollutants. The new, inexpensive sensors that have recently come onto the market cannot currently measure particles much smaller than 1 micron, nor can they characterize components of this pollution that individuals may be especially sensitive to (such as allergens), so electronic circuits, statistical techniques and software algorithms must be developed to estimate these pollutants from sensors as well as 3rd party data available over the Internet. Current home automation system and household appliances (both pollution sources and pollution mitigators) were designed without awareness of these new sensors, so new algorithms, circuitry, and techniques must be developed to incorporate this important data into their operation.

Historically, sensors capable of determining or even estimating air PM2.5 or PM10 levels have cost thousands or even tens of thousands of dollars. This expensive equipment measures pollutant levels in the traditional mass per unit volume (micrograms per cubic meter, typically), and most health studies that correlate pollutant exposure to health outcomes have used these units. More recently, particle counters have become available that measure particle counts within a size range range per unit volume. These two measures of pollution are not exactly the same; in particular, local humidity and precipitation conditions can swell the size of pollutants so that in more humid conditions they register higher particle counts, although the mass of the pollutants in the air is the same, presumably implying the same health impact in the body, and the same levels of dust when they settle on household furniture. The source of the pollutant (automobile versus cigarette smoke versus forest fire) also plays a role in the proper calibration of these particle count metrics against EPA and other health data expressed in mass per unit volume.

Particle counters have also not been inexpensive, but in the last few years very sensitive laser counters have become available for under $300. These been be accurately calibrated against EPA pollutant mass data under conditions of relatively constant pollutant source (typically automobile exhaust) and humidity/precipitation conditions. Moreover, in the last two years or so inexpensive 1-micron dust sensors have come onto the wholesale electronics market for under $10. At the time of writing these do not seem to have made into consumer models, however some hobbyist shops such as Wicked Devices' Air Quality Eggs are selling self-described ‘kits’ that incorporate the devices into near-consumer ready units. A number of companies have recently announced various plans to introduce more consumer-ready versions of these kinds of products over the next few years, but none of these proposals appears to adequately address the use of these sensors within the larger context of 3rd party Internet-available data, nor within the larger context of other devices and sensors accessible within the home through new home automation systems.

The calibration of the new, inexpensive sensors against the more accurate laser particle counters or EPA mass estimates is not well known, but the inventor was able to develop a mathematical model in a computer spreadsheet and then incorporate this into an iPhone prototype. Using noise filtration and linear regressions the inventor was able to establish a reasonable correlation between this $5 dust sensor's outdoor measurements and local EPA PM2.5 estimates in the good to moderate ranges, and the sensor was directionally correct going into the unhealthy ranges (and a non-linear calibration curve, such as a cubic spline, might need to be substituted.) Although incorporating 3rd party reference data from sources such as the EPA in the operation of software and control circuitry related to such sensors might seem useful, this solution does not appear to have been put into common use by any of the near-consumer-ready devices currently available in the United States to the inventor's knowledge, despite some evangelization by the inventor after the priority date of this application. The inventor's public iPhone prototype, published after the priority date of this application, is one exception.

Furthermore, current “near-consumer-ready” solutions do not provide a ready or obvious way to instruct or control other household devices. In addition to air purifiers, the obvious device needing control is the air filter commonly found in household forced air heating and cooling systems. These air filters are typically much less expensive to operate than air purifiers. As the inventor discovered, they are also not as capable in removing pollutants as air purifiers, and the correct threshold to activate and deactivate these air filters varies non-trivially from day-to-day. An electronic circuit or other means of combining information from the pollution sensors with internet or other information and then making a sophisticated decision about when to operate the air filter in the forced air system, and when to operate the air purifier instead, is needed. These existing devices also do not provide logic for controlling or scheduling polluting devices (e.g., dishwashers, gas dryers, gas ranges, furnaces, showers) to mitigate pollution. Nor do these devices and accompanying software provide a means of manipulating windows or heat exchange systems (or recommending such manipulation to the user) to reduce indoor air pollution under conditions where this might be appropriate.

Another shortcoming is that these devices and their accompanying software do not provide a means for estimating more precisely the different components of indoor air pollution, such as allergens. Such estimates might be inferred by combining precise 3rd party readings from a remote location (e.g., remote EPA readings) with crude by more local readings from a local sensor array.

Although the use of these inexpensive $5 1-micron particle counter/sensors has recently become common in hobbyist communities, interconverting and/or comparison this data with EPA data remains uncommon despite evangelization by the inventor after the priority date of this application. In particular, some of the more inexpensive sensors often return extremely poor/noisy data without the use of filtering methods developed by the inventor, such as the use of a simple moving average filter combined with a simple regression model known to those skilled in the art.

Thermostat and industrial climate control systems have existed for many years. However, only in the past 2 or 3 years have dust sensors become inexpensive enough where it would become practical to incorporate such a dust sensor into a common household thermostat for controlling the fan to efficiently reduce indoor dust by operating the fan for a longer time under control of an algorithm or circuit logic (either directly through circuitry within the thermostat itself or from a remote indoor dust sensor in communication via a control circuit incorporating home automation, computer, Bluetooth, WiFi, radio, direct computer network physical cabling, or similar means.)

Computer-controlled window opener/closers for use in home automation systems have existed for a few years, but only recently has it become practical to include a dust sensor in the circuit. The fact that indoor and outdoor air pollution levels very greatly over time in a city such as Los Angeles, and that indoor air pollution could benefit from strategic opening and closing of windows under computer control (either directly or involving manual human intervention) does not appear to have contemplated.

Fitness trackers that monitor physical exertion by the user using computer network means (e.g., Bluetooth or USB coupled with a smartphone or human computer) have become popular recently. The data quality produced by these inexpensive dust sensors has hitherto been too poor to contemplate use within a fitness tracker; the investor's improvement, in addition to reducing data noise through filtering techniques, is to combine with higher quality external data so that sensitive individuals' exposure to specific problematic pollutants (e.g., specific pollens) can be estimated or inferred even through the use of an inexpensive sensor that produces noisy data not by itself sufficiently specific for the pollutant or allergen of concern.

Household air filters have existed for many years, but even the most expensive systems, costing thousands of dollars, do not generally include linkages for communication or control from sensor-enabled home automation systems. Although such usage is envisioned, coordinating air filters with other household devices (most notably forced air ventilation systems, ventilation fans, and windows) as the inventor has described here has clearly not previously been envisioned; current practicers have difficulty just getting clean data from these cheap sensors, let alone using the new sensors now sometimes found within these devices to further coordinate with a climate system fan or operate a window.

Current systems do not envision the use of external ventilation when outdoor air quality is superior to indoor air quality, as may commonly happen after the operation of a typical dishwasher, shower, or indoor gas appliance. Current practice relies almost entirely on operating a simple air purifier, often continuously on the same setting. Current practice does not envision instead also coordinating automated windows, heat exchangers, climate control fans, or other devices to further rapidly relieve pollution, as becomes especially possible once 3rd party data, such as EPA information, is accessed over the Internet to facilitate intelligent, automated decision making regarding such device operation. Surprisingly, on a poor-quality day in a typical polluted city, a single or even multiple air purifiers on their typical settings may not be adequate to improve air quality to acceptable or desired levels, so this lack of intelligent marshaling of additional resources within the house becomes significant.

It would be advantageous to provide a way for combining local data from inexpensive pollution sensors with richer but less localized pollution, weather, and other data providers such as the US EPA. It would also be advantageous to provide ways for intelligently acting on this combined data in response to specific user needs, such as heightened sensitivity to specific allergens whose estimated presence emerges only from the combined, refined data. It would further be advantageous to provide ways to save money on air purification costs by allowing the inexpensive air filters found in common home forced air climate control systems to assist with home air purification by running longer, as appropriate, in response to pollution sensory inputs, external data, and algorithmic analysis to optimize operation of such inexpensive household air filters for maximum efficiency with respect to air quality improvement. It would further be advantageous to utilize common household ventilation controls, such as automatically or manually operated windows, to further assist in reducing household pollution in response to pollution sensory inputs combined with external data.

### SUMMARY OF THE INVENTION

In accordance with the present invention, there is provided software (including mathematical models implemented in software) and/or related electronic circuits that can be used to combine data from local, inexpensive dust sensors (particle counters) with Internet-available rich data on pollutants and weather (e.g., from governments source such as the US EPA, weather bureau) and optional household devices (appliances; alarm systems with knowledge of door and window states; polluting appliances such as dishwashers, ranges, dryers, and furnaces; air filters; and ventilation fans in common household heating/cooling systems and/or heat exchangers) to create a rich picture of the local environment, shape that environment through non-trivial control of said household appliances and ventilation systems to reduce buildup of household dust on surfaces or reduce sensitive individuals' exposure to specific pollutants, and monitor individuals' exposure to pollutants. The software might live in a smartphone (such as the inventors′iPhone prototype), related hardware devices (such as a pollution sensor communicating via bluetooth with the smartphone) or in heating/cooling control system such as a common household thermostat.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a plan view of a system for inexpensive characterization of air pollutants and inexpensive reduction of indoor dust.

For purposes of clarity and brevity, like elements and components will bear the same designations and numbering throughout the Figures.

### DESCRIPTION OF THE PREFERRED EMBODIMENT

The invention describes electronic circuits and/or software implementing mathematical, statistical, and/or computer models that can combine data from inexpensive indoor and outdoor dust sensors with rich data from Internet sources (such as detailed government data from high-end pollution and meteorological sensors in the same city, and data made available via Internet from other low-cost pollution sensor users), adaptive learning regarding the local environment (such as leakage of outdoor pollutants into home at different outdoor pollutant levels and in different states, such as an open or closed window, or ventilation an that is on or off) to create a rich mathematical or statistical description of levels of different pollutants in the local air. The system can automatically control several household systems, such as common household forced air heating/cooling ventilation fans to minimize indoor dust level and reduce housekeeping costs by minimizing dust buildup. The system can also extrapolate, by combining the data from expensive remote sensors on the Internet (e.g., government data) with local expensive sensors to alert sensitive individuals to levels of specific pollutants in their local outdoor or indoor environment that would be impossible to infer from either the inexpensive local sensor or the remote rich sensor alone.

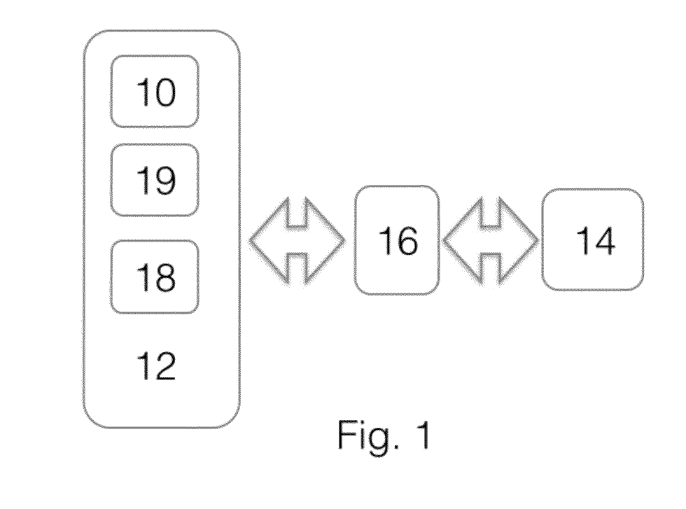

As previously mentioned, FIG. 1 is a plan view of the system for inexpensive characterization of air pollutants and inexpensive reduction of indoor dust. A local sensor array 10 is effectively interconnected to a pollution control system 16. This connection might consist of a direct wire interconnection (e.g., such as when the pollution control system 16 is a physical device such as a smart thermostat, and the local sensor array 10 is physically mounted on the same circuit board) or it might consist of a local wireless connection (e.g., a local home automation network operating using Zigbee, Z-wave, Bluetooth, Wifi, or other wireless local network standard as is known to those skilled in the art). In the preferred embodiment, the pollution control system 16 is also effectively interconnected (again, either directly or via wireless means) to a forced air climate control system 18, to allow it to allow it to run its fan longer, as needed, to utilize the system's air filter to mitigate air pollution under the control of a mathematical algorithm, as described herein. The preferred embodiment further includes an external ventilation control 19 to facilitate venting of polluted indoor air to less polluted outdoor air when the combination of sensors and 3rd party readings (or sensors alone) indicates that outdoor air is superior to indoor air according to the users' pollution preferences. The preferred embodiment is further effectively interconnected to a networked 3rd party data gatherer 14 so as to allow the mathematical or other algorithm within the control unit access to 3rd party remote pollution data (e.g., outdoor PM2.5 pollution and pollen levels as provided by the EPA) as described herein. In the preferred embodiment, the local sensor array 10, forced air climate control system 18, and external ventilation control 19 are components of a larger home automation system 12 with additional sensors and devices as described herein, and are thus indirectly interconnected with the pollution control system 16, but in other embodiments may be more directly interconnected, such as having the pollution sensors mounted directly within a smart thermostat's circuitry as previously described.

The inventor developed an iPhone app prototype that is able to convert data from this inexpensive sensor, apply the noise-reduction filtering and combine it in a simple linear model with information about local humidity and precipitation, to generate numeric values comparable to local EPA outdoor air measurements. These inexpensive dust sensors are still too large (and too new) at time of writing to be conveniently incorporated directly into sensor-laden devices such as smartphones, smartwatches or other wearable devices (Google Glass), but they are small enough to be incorporated into a separate, bluetooth (or other communication means) device that could be carried with the person (e.g., in a handbag) to track pollutant exposure in a manner similar to how current fitness trackers (e.g., FitBit) are used. The tracker would then sync with a computational device (such as a smartphone) using Bluetooth, Wifi, cable, or other communication systems.

It is important to note that indoor pollutant levels are often very different from outdoor pollutant levels. Therefore, local indoor pollutant sensors (such as the inexpensive 1-micron sensor, or the inexpensive 0.5-micron laser particle counter) are needed, as indoor pollution levels cannot be accurately obtained over the Internet from a 3rd party source such as the US EPA.

In the inventor's experiments at his home in urban Los Angeles, Calif., in just two weeks outdoor 0.5-micron counts per 0.1 cubic fey” range, subsequently confirmed in correspondence with local air pollution authorities despite a malfunction preventing the information from appear in their web-published data. According to the WHO organization, average annual exposures above 10 micrograms per cubic meter and 24-hour averaged exposures above 24 micrograms per cubic meter in the PM2.5 range are associated with negative health outcomes such as increased incidence of lung cancer. This means someone living outdoors 24/7 in Los Angeles would easily be at elevated risk for lung cancer. The EPA “unhealthy” is well above even the 24 microgram per cubic meter WHO warning level, although it not stay there for 24 hours.)

In contrast, the inventor's filtered air are was typically under 100 0.5-micron counts (per 0.1 cubic feet), or nearly an order of magnitude better than even the best outdoor Los Angeles air, and nearly three orders of magnitude better than the worst Los Angeles air the inventor observed in his two week experiment. However, as the EPA notes, indoor air can be 10× worse than outdoor air. The inventor observed that operation of his ordinary dishwasher unit caused indoor particle counts to increase nearly two orders of magnitude from 50 to over 3000, or about 5× worse than the polluted Los Angeles air on a good day. Similarly, operation of the inventor's shower, vacuum cleaner, gas range or gas dryer also caused indoor quality to substantially deteriorate. (The EPA has noted that showers and dishwashers are some of the worst indoor pollution sources.) In addition to being unhealthy, these indoor (and outdoor) pollution sources contribute to indoor dust buildup (and housekeeping expenses) and do not vary evenly by time, but rather occur in spikes and bursts due to the substantial variance in outdoor air pollution as well as with typical non-continuous use patterns of devices causing indoor pollution.

The inventor experimented with several ways of purging the indoor air pollution created by his dishwasher and gas appliances. On good days in Los Angeles, the fastest, cheapest, and most efficient remedy was simply to open the window. This would bring indoor particle counts down to the outdoor level, which was sometimes significantly below that created by the dishwasher, at which point the window would be closed to facilitate further de-pollution by the inventor's air cleaners to well below the outdoor air pollution levels. Automatic window openers/closers exist on the market for under $500; these can allow operation of the window from a computer and sometimes include environmental sensors such as rain detectors that trigger an automatic window close (no units currently factor in indoor or outdoor dust levels.) The author envisions an electronic home control system that would monitor indoor dust levels (by means of aforementioned inexpensive dust sensor means) and outdoor dust awareness (either by inexpensive outdoor dust sensors, or by obtaining the information over the Internet) to detect indoor pollution (e.g., caused by a dishwasher) or anticipate the pollution (by notification from the appliance that it is about to operate), open the window whenever the sensed or anticipated indoor air pollution exceeds the sensed or modeled outdoor air pollution, and close the window once indoor air quality has been equalized with outdoor air quality. In many cultures (e.g., Europe) it is common ritual to purge the indoor air by briefly opening all of the windows; however this is done without sensor telemetry.

The inventor's experiments show that these rituals would be better controlled via computer software (e.g., a motorized solution, or a simple smartphone app that simply tells the operator when to open and close the windows.) Most of the time, indoor air quality far exceeds outdoor air quality, so this daily ritual is counterproductive at these times. However, at certain times during the day, such as after the operating of certain appliances, indoor air quality can be much worse than outdoor air quality. Through the use of sensors, home automation systems, and external 3rd party data telemetry can inform window opening and closing to optimize indoor air quality. The investor's system envisions window opening and closing mechanisms (either motorized or through a message to the operator) in combination with computer models and/or a second outdoor sensor to achieve such indoor air quality optimization in an inexpensive way. This system works in warm climates (such as Los Angeles), but in the rest of the country this system could be also factor in outdoor temperatures (and thus model a trade off between heating/cooling costs and air quality) or the operation of a heat exchanger or other cold climate ventilation mechanisms to achieve a similar effect. The author has prototyped these inventions using computer spreadsheets, as well as an iPhone app prototype that converts indoor air particle counts to EPA-data equivalents. This prototype can detect when indoor air quality is worse than outdoor air quality, and can display a message to the user to the effect (and suggest opening a window or turning off the appliance via a popup message). This iPhone app could easily be modified to control a home automation system 12 to directly open and close the window (perhaps taking weather conditions, such as rain or temperature, into account in the window opening and closing decision, as well as security considerations, such as querying the home security system as to whether the homeowner was home or not.)

A more significant observation was the impact of the investor's heating/cooling fan on indoor air quality. The author has a common forced air heating/cooling system, which includes a separate control for manually forcing the fan to operate even when the system is not heating. The forced air system includes a common, inexpensive air filter for such systems (MERV 13) placed in the intake duct.

When indoor pollution was high (e.g., operation of the dish washer), and yet below the level of outdoor air pollution, the most efficient way to reduce indoor air pollution was by activating the forced air fan, which sucked air through the inventor's MERV 13 filter. This rapidly reduced indoor pollution to some level M, where M depended greatly on the outdoor air pollution level, and where M was still well above the ideal level of indoor air pollution recommended for some sensitive individuals (and still contributing to dust-related equipment failure and household cleaning bills). The forced air filter was able to filter the air, but its operation apparently created a suction effect in the home which brought increased levels of polluted air from outside the home.

Once air pollution in the home was below the outside level, the inventor operated the forced air fan until some level M was achieved. Surprisingly, after level M was achieved, continuing to operate the forced air fan was inefficient, and was actually increasing the level of indoor air pollution by bringing in additional polluted air from outside. Therefore, the author would turn off the forced air fan at level M, and allow inexpensive household air cleaners (which were previously inefficient or slow to act on the more polluted indoor air) to operate. The inventor was able to use indoor dust measurements, outdoor dust measurements at different times, and knowledge of whether the ventilation fan was operating or not to establish a simple linear regression or cubic spline curve that can compute M from a given outdoor dust measurement level.

One embodiment of the invention incorporates a modification to the electronic control circuits commonly found household heating/cooling thermometers that uses a linear, cubic spline, or similar mathematical means known to those skilled in the art to estimate M from current outdoor dust measurements as well as past observation of indoor dust levels, and operate the ventilation fan to improve indoor air quality. In practice, the thermostat might keep the fan on longer than current thermostats do after heating/cooling has ceased, so as to enable additional air filtration, but only as long as such operation is efficiently contributing to a reduction in indoor air pollution. A simple embodiment might continue to operate the fan, and deduce, measure, or detect the current level M by detecting when pollution mitigation due to the forced air fan appears to have plateaued.

The invention might further compromise manual notification or home automation means to adjust air filter systems in response to sensor data, and coordinate with other control means just described (window control means, heat exchanger control means, forced air ventilation control means, interrogation of home security systems for window/door open/closed and homeowner present/not-present status, interrogation of home automation systems or appliance systems for polluting and ventilating appliance status.)

The inventor further notes that not all PM2.5 pollutants are equally bad. Sensitive individuals may not be sensitive to all PM2.5 pollutants equally, but may have an especially sensitive to a given subset of such pollutants (e.g., specific pollens) that may very greatly from day to day. Consequently, the inexpensive dust sensors described herein, when used alone, cannot estimate a sensitive individual's exposure to specific allergens or other problematic pollutants. Moreover, although detailed information on these pollutant levels at a somewhat distant location may be available over the Internet (due to expensive monitoring performed at a distance by government agencies such as EPA), this Internet-available information would not accurately characterize the sensitive individual's local indoor air for many of the reasons just described.

One embodiment of the invention includes computer, algorithmic, or electronic circuit means for estimating the rate of leakage of outdoor air pollutants into indoor air (e.g., similar to methods for computing M as described in the modified thermostat invention above, which can be found by measuring the change in indoor dust levels over time against different outdoor dust levels under reasonable constant circumstances, such as the operation or non-operation of the heating/cooling ventilation fan. M for a given outdoor pollution level is directly related to the pollutant leakage rate while the ventilation fan is operating.) The computer means may incorporate history tracking means (such as querying the home security system about the open/closed status of external doors and windows, ventilation fan operation history, appliance operation history) and correlation means (e.g., simple statistical regression as would be known to one skilled in computer modeling or statistical modeling).

This invention would then combine an indoor air dust sensor, an outdoor dust measurement reading (either another sensor or 3rd party Internet-data based) and aforementioned outdoor air pollutant leakage rate estimate means (which might be estimated to vary over time due to ventilation fan status or opening and closing of doors and windows) to transform government or other Internet-based information about specific particulate pollutants (e.g., specific pollens) to create detailed estimates of specific pollutant levels in the home using only the inexpensive dust sensor and the detailed 3rd party Internet-based pollution data from one or more sensors (and possibly inputs on humidity and precipitation that affect particle counts).

Three primary uses for the invention are envisioned: (1) computer, home automation, manual, and/or environmental control means for the reduction of indoor air pollutants, both for reasons of health in normal individuals and to reduce household cleaning expenses and/or time and (2) tracking of pollutant exposure for individuals of normal sensitivity who live in polluted environments to enable them to manage their 24-hour and annual average exposures in accordance with current WHO recommendations and (3) tracking of sensitive individuals exposure to specific pollutants using only the inexpensive sensor and the remote Internet-data in a way that cannot currently be done using either the sensor or the data alone.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A system for inexpensive characterization of air pollutants and inexpensive reduction of indoor dust for characterizing and mitigating indoor air pollution, comprising:
means for sensing the local (e.g. in-home) environment (for e.g. pm2.5 dust pollution);
means for gathering 3rd party data (e.g., epa outdoor pm2.5 dust pollution, precise data on air composition of different pollutions, weather or wind conditions, models of pollutant distribution) via networked means (e.g., internet, cellular data, etc.); and
means for integrating data from local sensor array, networked 3rd party data gatherer and optional home automation system to determine local conditions (e.g., using mathematical models and/or algorithms) within an electronic device or software application (e.g., smart thermostat, smartphone app or home automation app) and optionally send commands to an optional home automation system (e.g., schedule dish washer), effectively interconnected to said means for gathering 3rd party data (e.g., epa outdoor pm2.5 dust pollution, precise data on air composition of different pollutions, weather or wind conditions, models of pollutant distribution) via networked means (e.g., internet, cellular data, etc.), and effectively interconnected to said means for sensing the local (e.g. in-home) environment (for e.g. pm2.5 dust pollution).

2. The system for inexpensive characterization of air pollutants and inexpensive reduction of indoor dust in accordance with claim 1, wherein said means for sensing the local (e.g. in-home) environment (for e.g. pm2.5 dust pollution) comprises a pm2.5 sensor as part of a local sensor array.

3. The system for inexpensive characterization of air pollutants and inexpensive reduction of indoor dust in accordance with claim 1, wherein said means for gathering 3rd party data (e.g., epa outdoor pm2.5 dust pollution, precise data on air composition of different pollutions, weather or wind conditions, models of pollutant distribution) via networked means (e.g., internet, cellular data, etc.) comprises a network connection (e.g. internet or cellular data), access to remote and/or 3rd party detailed pollutant and/or weather data via the networked 3rd party data gatherer.

4. The system for inexpensive characterization of air pollutants and inexpensive reduction of indoor dust in accordance with claim 1, wherein said means for integrating data from local sensor array, networked 3rd party data gatherer and optional home automation system to determine local conditions (e.g., using mathematical models and/or algorithms) within an electronic device or software application (e.g., smart thermostat, smartphone app or home automation app) and optionally send commands to an optional home automation system (e.g., schedule dish washer) comprises a mathematical model (e.g., regression) to non-trivially combine information within pollution control system.

5. A system for inexpensive characterization of air pollutants and inexpensive reduction of indoor dust for characterizing and mitigating indoor air pollution, comprising:
a pm2.5 sensor as part of a local sensor array, for sensing the local (e.g. in-home) environment (for e.g. pm2.5 dust pollution);
a network connection (e.g. internet or cellular data), access to remote and/or 3rd party detailed pollutant and/or weather data via the networked 3rd party data gatherer, for gathering 3rd party data (e.g., epa outdoor pm2.5 dust pollution, precise data on air composition of different pollutions, weather or wind conditions, models of pollutant distribution) via networked means (e.g., internet, cellular data, etc.); and
a mathematical model (e.g., regression) to non-trivially combine information within pollution control system, for integrating data from local sensor array, networked 3rd party data gatherer and optional home automation system to determine local conditions (e.g., using mathematical models and/or algorithms) within an electronic device or software application (e.g., smart thermostat, smartphone app or home automation app) and optionally send commands to an optional home automation system (e.g., schedule dish washer), effectively interconnected to said networked 3rd party data gatherer, and effectively interconnected to said local sensor array.

6. The system for inexpensive characterization of air pollutants and inexpensive reduction of indoor dust as recited in claim 5, further comprising:
a home automation wireless system (e.g., zigbee, z-wave, wifi, bluetooth), devices with sensors (e.g., air purifier, alarm system with door/window status), polluting device controls (e.g., dishwasher, gas range, gas dryer, shower controls), mitigating device controls (e.g., window or heat exchanger controls) home automation system, for gathering data from in-appliance sensors (e.g., sensors in an air purifier, door/window status from a home alarm) and controlling polluting and mitigating devices (e.g., controlling dishwasher, gas range, gas dryer, furnace to reduce pollution, opening or closing windows or heat exchanger to mitigate pollution), effectively interconnected to said pollution control system.

7. The system for inexpensive characterization of air pollutants and inexpensive reduction of indoor dust as recited in claim 5, further comprising:
a common, inexpensive air filter for forced air systems (e.g., furnace and/or ac filter), common forced air fan and control for a forced air climate control system, for utilizing the built-in inexpensive air filter and fan commonly found in home forced air systems to mitigate dust pollution by running the fan longer, under the control of an algorithm, effectively interconnected to said pollution control system.

8. The system for inexpensive characterization of air pollutants and inexpensive reduction of indoor dust as recited in claim 5, further comprising:
an external ventilation control, for allowing the control system to externally vent polluted indoor air if it is determined that outdoor air is less polluted (e.g., via motorized window, heat exchanger, other vent control mechanism, or manually prompting of a user), effectively interconnected to said pollution control system.

9. The system for inexpensive characterization of air pollutants and inexpensive reduction of indoor dust as recited in claim 6, further comprising:
a common, inexpensive air filter for forced air systems (e.g., furnace and/or ac filter), common forced air fan and control for a forced air climate control system, for utilizing the built-in inexpensive air filter and fan commonly found in home forced air systems to mitigate dust pollution by running the fan longer, under the control of an algorithm, effectively interconnected to said pollution control system.

10. The system for inexpensive characterization of air pollutants and inexpensive reduction of indoor dust as recited in claim 6, further comprising:
an external ventilation control, for allowing the control system to externally vent polluted indoor air if it is determined that outdoor air is less polluted (e.g., via motorized window, heat exchanger, other vent control mechanism, or manually prompting of a user), effectively interconnected to said pollution control system.

11. The system for inexpensive characterization of air pollutants and inexpensive reduction of indoor dust as recited in claim 7, further comprising:
an external ventilation control, for allowing the control system to externally vent polluted indoor air if it is determined that outdoor air is less polluted (e.g., via motorized window, heat exchanger, other vent control mechanism, or manually prompting of a user), effectively interconnected to said pollution control system.

12. The system for inexpensive characterization of air pollutants and inexpensive reduction of indoor dust as recited in claim 9, further comprising:
an external ventilation control, for allowing the control system to externally vent polluted indoor air if it is determined that outdoor air is less polluted (e.g., via motorized window, heat exchanger, other vent control mechanism, or manually prompting of a user), effectively interconnected to said pollution control system.

13. A system for inexpensive characterization of air pollutants and inexpensive reduction of indoor dust for characterizing and mitigating indoor air pollution, comprising:
a pm2.5 sensor as part of a local sensor array, for sensing the local (e.g. in-home) environment (for e.g. pm2.5 dust pollution);
a home automation wireless system (e.g., zigbee, z-wave, wifi, bluetooth), devices with sensors (e.g., air purifier, alarm system with door/window status), polluting device controls (e.g., dishwasher, gas range, gas dryer, shower controls), mitigating device controls (e.g., window or heat exchanger controls) home automation system, for gathering data from in-appliance sensors (e.g., sensors in an air purifier, door/window status from a home alarm) and controlling polluting and mitigating devices (e.g., controlling dishwasher, gas range, gas dryer, furnace to reduce pollution, opening or closing windows or heat exchanger to mitigate pollution);
a network connection (e.g. internet or cellular data), access to remote and/or 3rd party detailed pollutant and/or weather data via the networked 3rd party data gatherer, for gathering 3rd party data (e.g., epa outdoor pm2.5 dust pollution, precise data on air composition of different pollutions, weather or wind conditions, models of pollutant distribution) via networked means (e.g., internet, cellular data, etc.);
a mathematical model (e.g., regression) to non-trivially combine information within pollution control system, for integrating data from local sensor array, networked 3rd party data gatherer and optional home automation system to determine local conditions (e.g., using mathematical models and/or algorithms) within an electronic device or software application (e.g., smart thermostat, smartphone app or home automation app) and optionally send commands to an optional home automation system (e.g., schedule dish washer), effectively interconnected to said networked 3rd party data gatherer, effectively interconnected to said home automation system, and effectively interconnected to said local sensor array;
a common, inexpensive air filter for forced air systems (e.g., furnace and/or ac filter), common forced air fan and control for a forced air climate control system, for utilizing the built-in inexpensive air filter and fan commonly found in home forced air systems to mitigate dust pollution by running the fan longer, under the control of an algorithm, effectively interconnected to said pollution control system; and
an external ventilation control, for allowing the control system to externally vent polluted indoor air if it is determined that outdoor air is less polluted (e.g., via motorized window, heat exchanger, other vent control mechanism, or manually prompting of a user), effectively interconnected to said pollution control system.
